(19) United States
(12) Patent Application Publication
(10) Pub. No.: US 2006/0030743 A1
(43) Pub. Date: Feb. 9, 2006

(54) Catalyst and method for producing alcohol by hydrogenation on said catalyst

(76) Inventors: Markus Schubert, Lubwigshafen (DE); Sylvia Huber-Dirr, Zwingenberg (DE); Michael Hesse, Worms (DE); Jochen Pfeifer, Ilbesheim (DE); Rolf-Hartmuth Fischer, Heidelberg (DE); Markus Rosch, Oppenheim (DE); Nils Bottke, Mannheim (DE); Alexander Weck, Freinsheim (DE); Gunther Windecker, Ludwigshafen (DE); Gunnar Heydrich, Limburgerhof (DE)

Correspondence Address: CONNOLLY BOVE LODGE & HUTZ LLP, SUITE 800, 1990 M STREET NW, WASHINGTON, DC, 20036-3425, US

(21) Appl. No.: 10/534,457

(22) PCT Filed: Nov. 6, 2003
(86) PCT No.: PCT/EP03/12378

§ 371 Date: May 11, 2005

(30) Foreign Application Priority Data
Nov. 11, 2002 (DE) 102 52 280.4

(51) Int. Cl. B01J 23/656; C07C 29/14; C07C 29/136

(52) U.S. Cl. 568/880; 502/339

(57) ABSTRACT

The invention relates to a catalyst containing from 0.1 to 20 mass % rhenium and 0.05 to 10 mass % platinum in relation to the total mass thereof. The inventive method for producing a support for said catalyst consists in a) treating a support which can be eventually pre-treated with the aid of a solution of rhenium compound, b) drying and annealing said support at a temperature ranging from 80 to 600° C., and c) impregnating the support with a solution of platinum compound and in drying it. The inventive method for producing alcohol by catalytic hydrogenation of carbonyl compound on said catalyst is also disclosed.

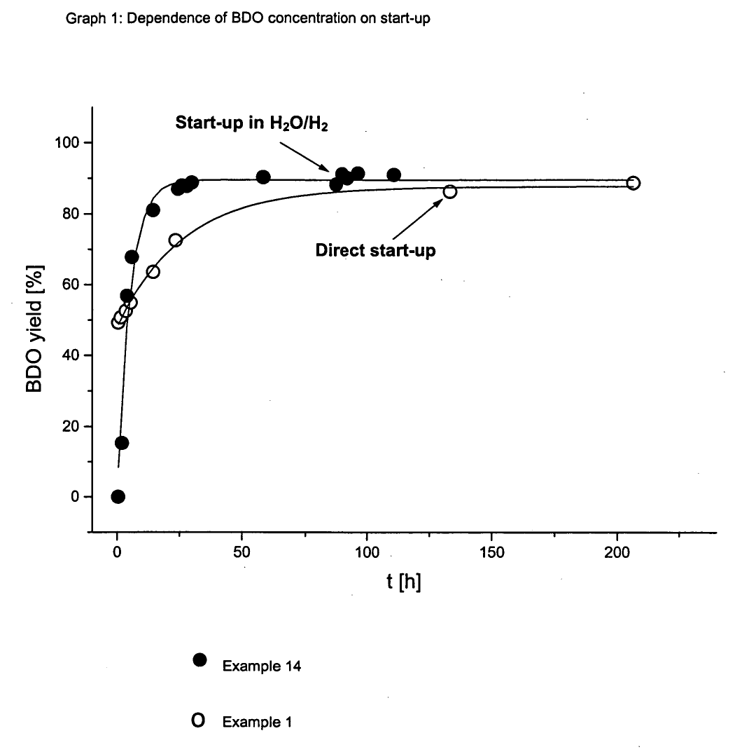

The invention relates to a process for preparing alcohols by hydrogenating compounds containing carbonyl groups over supported rhenium catalysts and also to the improved supported rhenium catalysts.

The industrial hydrogenation of reactants containing carbonyl groups, such as aldehydes, ketones, carboxylic acids, carboxylic anhydrides and esters, over rhenium catalysts to alcohols is known per se.

DE-A 2 519 817 describes catalysts for the hydrogenation of maleic anhydride, maleic acid or mixtures thereof, which at the same time contain elements of transition groups VII and VIII of the Periodic Table of the Elements. The catalysts preferably comprise rhenium and palladium which, according to the examples, are preferably applied at the same time to the support in the course of the catalyst preparation. According to the disclosure content of DE-A 2 519 817, it is also possible to apply the palladium compound to the support first.

After a drying step, this results in supported palladium-rhenium catalysts whose activity in hydrogenations of compounds containing carbonyl groups is so low that it is necessary at the same time to use high pressures and high temperatures of from 215 to 230° C. As a result of high energy and material costs, carrying out the hydrogenations at high pressures and high temperatures is of low economic viability. In addition, the corrosivity, especially when using carboxylic acid solutions, increases under these conditions.

In some hydrogenation reactions, for example the hydrogenation of carboxylic acids or their derivatives to alcohols, palladium-rhenium catalysts also have the disadvantage that the palladium promotes side reactions such as ether formation.

The avoidance of ethers as a by-product of industrial hydrogenation processes is the aim of DE-A 100 09 817. The catalysts prepared by simultaneous application of rhenium and palladium compounds to an activated carbon support and subsequent drying steps prove to be substantially more selective. However, the catalysts obtained are so inactive that very high noble metal loadings of the support of more than 8% by weight of platinum and more than 14.5% by weight of rhenium are required to achieve the desired activity, which makes the process disclosed in DE-A 100 09 817 appear uneconomic overall.

It is an object of the present invention to provide a process for catalytically hydrogenating carbonyl compounds to alcohols and also catalysts which do not have the disadvantages of the prior art. The catalysts should also be suitable to hydrogenate carbonyl compounds in the liquid phase to alcohols in high yield and selectivity.

We have found that this object is achieved by a catalyst comprising from 0.1 to 20% by weight of rhenium and from 0.05 to 10% by weight of platinum, based on the total mass of the catalyst, on a support, obtainable by a process in which
a) the optionally pretreated support is treated with a solution of a rhenium compound,
b) dried and heat-treated in a reductive atmosphere at from 80 to 600° C.,
c) impregnated with a solution of a platinum compound and dried again.

The support or support material used in the catalyst according to the invention is generally a metal oxide such as the oxides of aluminum and titanium, zirconium dioxide, hafnium dioxide, silicon dioxide, optionally pretreated activated carbon or else optionally pretreated graphitic carbon supports, nitride, silicide, carbide or boride. The pretreatment mentioned may be an oxidative pretreatment, as described, for example, in EP-A 848 991. A nonoxidative pretreatment of the support material, for example with phosphoric acid, is disclosed by DE-A 100 09 817. Preference is given to using supports of titanium dioxide, zirconium dioxide, hafnium dioxide, optionally pretreated activated carbon and/or graphitic carbon.

The rhenium component used is customarily (NH₄)ReO₄, Re₂O₇, ReO₂, ReCl₃, ReCl₅, Re(CO)₅Cl, Re(CO)₅Br or Re₂(CO)₁₀, although this list is not intended to be exclusive. Preference is given to using Re₂O₇.

In addition to rhenium, preference is given to also applying platinum to the catalyst. The platinum may be applied, for example, as platinum powder, oxide, oxide hydrate, nitrate, platinum(II) or (IV) chloride, hydrogen hexachloroplatinate(IV), platinum(II) or -(IV) bromide, platinum(II) iodide, cis- or trans-diaminoplatinum(II) chloride, cis- or trans-diaminoplatinum(IV) chloride, diaminoplatinum(II) nitrite, ethylenediaminoplatinum(II) chloride, tetraaminoplatinum(II) chloride or chloride hydrate, tetraaminoplatinum(II) nitrate, ethylenediaminoplatinum(II) chloride, tetrakis(triphenylphosphine)platinum(0), cis- or trans-bis(triethylphosphine)platinum(II) chloride, cis- or trans-bis(triethylphosphine)platinum(II) oxalate, cis-bis(triphenylphosphine)platinum(II) chloride, bis(triphenylphosphine)platinum(IV) oxide, (2,2′-6′,2″-terpyridine)platinum(II) chloride dihydrate, cis-bis(acetonitrile)platinum dichloride, cis-bis(benzonitrile)platinum dichloride, platinum(II) acetylacetonate, (1c,5c-cyclooctadiene)platinum(II) chloride or bromide, platinum nitrosylnitrate, preferably as platinum oxide or nitrate, more preferably as platinum nitrate, although this list is not intended to be exclusive.

Rhenium (calculated as the metal) is present in an amount of from 0.1 to 20% by weight, preferably from 0.5 to 13% by weight, more preferably from 2 to 7% by weight, based on the total mass of the catalyst composed of catalytic active composition and support material. The catalyst according to the invention comprises palladium (calculated as the metal) in an amount of from 0.05 to 10% by weight, preferably from 0.1 to 8% by weight, more preferably from 0.3 to 4% by weight, based on the total mass of the catalyst composed of catalytic active composition and support material.

The weight ratio of rhenium to platinum (calculated as the metals) is in the range of 0.01-100, preferably from 0.05 to 50, more preferably from 0.1 to 10.

Further elements may also be present on the catalyst. Examples include Sn, Zn, Cu, Ag, Au, Ni, Fe, Ru, Mn, Cr, Mo, W and V. These elements modify substantially the activity and selectivity (hydrogenolysis products) of the catalyst, but are not essential. Their weight ratio to Re may be from 0 to 100, preferably from 0.5 to 30, more preferably from 0.1 to 5.

The application of the active components rhenium (Re) and, if desired, platinum (Pt) can be carried out by impregnation, each in one or more steps within the process stages a) or c), with a solution in water, alcohol or another organic solvent of the particular dissolved compounds (for example salts, oxides, hydroxides), impregnation with a solution of dissolved oxidic or metallic colloid of the active component, equilibrium adsorption, in one or more steps, of the salts dissolved in aqueous or alcoholic solution, or equilibrium adsorption of dissolved metallic or oxidic colloid, on the pretreated activated carbon.

The impregnation itself may be effected either by saturating with the solution or else by spraying them on. The support can also be impregnated with solutions of salts which readily decompose thermally, for example with nitrates, or complexes which readily decompose thermally, for example carbonyl or hydrido complexes of the catalytically active elements, and the support saturated in this way heated to temperatures of from 300 to 600° C. for the purposes of thermal decomposition of the adsorbed metal compounds. This thermal decomposition can be carried out under a protective gas atmosphere. Suitable protective gases are, for example, nitrogen, carbon dioxide, hydrogen or the noble gases.

The drying steps in stages a) to c) are generally carried out at from room temperature to 120° C. Preference is given to heat-treating at from 150 to 450° C., more preferably at from 250 to 375° C.

In this application, a reductive atmosphere is an atmosphere which comprises at least a portion of a reducing gas such as ammonia, hydrazine, C₂- to C₆-olefin, carbon monoxide and/or hydrogen, and preference is given to hydrogen. The reducing atmosphere used is more preferably a hydrogen/nitrogen mixture whose hydrogen content is from 10 to 50% by weight.

It has proven economically favorable and is therefore preferred, after step b) of the process, to passivate the catalyst blank obtained with an oxygenous gas. The temperature for the passivation procedure is not critical, but should not exceed 120° C. Passivation is generally effected at room temperature.

The catalysts are customarily activated before use. This activation can be effected by applying a reducing gas atmosphere to the catalyst. Preference is given to activating with the aid of hydrogen. The activation temperature is customarily at 100-500° C., preferably 150-450° C., more preferably 200-400° C. Alternative reduction methods are the reduction of metallic components by contacting with a liquid reducing agent such as hydrazine, formaldehyde or sodium formate. Contacting with the liquid reducing agent is customarily effected at temperatures between 10 and 100° C. Particular preference is given to contacting at temperatures between 20 and 80° C.

Suitable starting materials for the hydrogenation are generally carbonyl compounds which may contain additional C—C double or triple bonds. Examples of aldehydes are propionaldehyde, butyraldehyde, crotonaldehyde, ethylhexanal, nonanal and glucose. Examples of carboxylic acids are succinic acid, fumaric acid, maleic acid, glutaric acid, adipic acid, 6-hydroxycaproic acid, octanedioic acid, dodecanedioic acid, 2-cyclododecylpropionic acid and saturated or unsaturated fatty acids. Esters include esters of the abovementioned acids, for example as the methyl, ethyl, propyl or butyl ester, and lactones, e.g. gamma-butyrolactone, delta-valerolactone or caprolactone, can also be used. It is also possible to use anhydrides such as succinic anhydride or maleic anhydride. Preferred starting materials are succinic acid, maleic acid, glutaric acid, adipic acid, 2-cyclododecylpropionic acid, succinic anhydride, maleic anhydride and also the esters of these acids and gamma-butyrolactone. It will be appreciated that it is also possible to use mixtures of aldehydes, carboxylic acids, esters, anhydrides and/or lactones, preferably mixtures of carboxylic acids, more preferably mixtures of maleic acid, glutaric acid and/or adipic acid.

The compounds to be hydrogenated may be hydrogenated without solvent or in solution. Useful solvents are preferably intermediates and hydrogenation products themselves, for example gamma-butyrolactone, or materials are used such as alcohols such as methanol, ethanol, propanol or butanol, and also suitable are ethers such as THF or ethylene glycol ethers. A preferred solvent is water, especially in the hydrogenation of carboxylic acids.

The hydrogenation may be performed in the gas or liquid phase, in one or more stages. In the liquid phase, both the suspension and the fixed bed method are possible. Preference is given to carrying it out in the liquid phase. In the case of exothermic reactions, the heat can be removed by external coolants (for example tubular reactor). Evaporative cooling in the reactor is also possible, in particular when hydrogenation is effected without product recycling. In the case of product recycling, one possibility is a cooler in the recycle stream.

The scope of the invention includes the recognition that particularly advantageous results are achieved in the preparation of alcohols by catalytic hydrogenation of carbonyl compounds in the process according to the invention when the hydrogenation reactor charged with the catalyst is started up under hydrogenation conditions with water or a dilute aqueous solution of the carbonyl compound which may also contain other solvents which are inert under the reaction conditions. In this context, start-up refers to putting the catalyst into service. This may be the first time a reactor is put into service, or else when it is put back into service, for example after plant shutdown for the purpose of maintenance, with fresh or regenerated catalyst. The start-up is carried out under the reaction conditions of the hydrogenation with respect to pressure and temperature and generally takes from 30 min to 20 h, preferably from 1 to 5 h. The dilute aqueous solution of the carbonyl compound contains a maximum of 5% by weight thereof.

The hydrogenation is customarily carried out at from 80 to 210° C., preferably at from 80 to 170° C., more preferably at from 100 to 150° C. Hydrogenation is effected customarily at a reaction pressure between 20 and 230 bar, preferably 50 and 220 bar, more preferably 70 and 160 bar. The catalyst hourly space velocity is dependent upon the noble metal concentration of the catalyst, but is generally higher than 0.05 g/m·h.

The alcohols obtained in the process according to the invention are used, for example, as solvents and intermediates. Diols such as butanediol find use as the diol components in polyesters.

The process according to the invention is illustrated by the examples which follow.

### EXAMPLES

The quoted contents of the individual components in the hydrogenation effluents have been determined by gas chromatography. Unless otherwise stated, they are calculated without solvent.

Abbreviations Used

MA=maleic acid, MAN=maleic anhydride, SA=succinic acid, SAN=succinic anhydride, GBL=gamma-butyrolactone, BDO=1,4-butanediol, THF=tetrahydrofuran, Bu-OH=butanol, carbon balance: values smaller than 100% result in particular from gaseous by-products which are not registered by the analysis, mainly n-butane. In the event of relatively high MA/MAN or SA/SAN contents, the analysis of these components is distorted, so that excessively high SA/SAN values are obtained in this case, which leads to a carbon balance of distinctly above 100%. The analysis of the remaining components is unaffected. In the examples, excessively high SA/SAN values were not corrected, but quoted as measured.

### Example 1

### Catalyst A

ZrO₂ extrudates (XZ 16509 from Norton, Chem Process Prod. Corp., Akron (Ohio), USA, BET surface area 82 m²/g, pore volume 0.28 cm³/g) were crushed to 1.2-2 mm spall. 200 g of this support material were treated with 40 ml of an aqueous solution of Re₂O₇ (10% by weight of Re₂O₇) with the addition of a further 140 ml of water for 3 h. The material was then dried at 111° C. and a pressure of 30-50 mbar for 2 h. Afterwards, it was heated under 200 l (STP)/h of N₂ at 150° C. over 30 min and kept at this temperature for 60 min. Subsequently, a mixture of 100 l (STP)/h of H₂ and 100 l (STP)/h of N₂ was fed in for 60 min, the material was heated to 300° C. within 30 min and the conditions were maintained for a further 3 h. The precatalyst was then cooled to 50° C. in this reducing atmosphere and subsequently in pure N₂ (200 l (STP)/h) to room temperature. The material was passivated with a mixture of 10 l (STP)/h of O₂ in 180 l (STP)/h of N₂ over 14 h and finally saturated with 39.24 g of a platinum nitrate solution (16.25% by weight of Pt) in water per 160 ml. After a contact time of 3 h, drying was effected at 100° C. and a pressure of from 30 to 50 mbar for 2 h. The finished catalyst was reduced. Catalyst A contained 2.9% by weight of rhenium and 2.9% by weight of platinum.

### Examples 2 and 3, Comparative Example 1

25 ml of catalyst A were installed into a continuous tubular reactor. The feed (reactant mixture) consisted of a maleic acid solution (10% by weight of maleic acid) and 50 l (STP)/h of hydrogen. The hydrogenation was carried out at 160° C. and 80 bar. The liquid effluent was analyzed by means of gas chromatography. The values in table 1 are each quoted as yields in % per mole of maleic acid.

TABLE 1

| | Catalyst hourly | MA | | | | | | Carbon |
|---|---|---|---|---|---|---|---|---|
| | space velocity | conversion | BDO | GBL | THF | BuOH | SA/SAN | balance |
| Ex. | [g_{(MA)}/ml_{(cat)} · h] | [%] | [%] | [%] | [%] | [%] | [%] | [%] |
| C1 | 0.05 | 100 | 58.6 | 0.0 | 0.7 | 17.9 | 0.0 | 79.4 |
| 2 | 0.1 | 100 | 88.8 | 0.1 | 0.1 | 8.7 | 0.0 | 99.5 |
| 3 | 0.2 | 100 | 72.5 | 14.4 | 0.9 | 6.3 | 0.0 | 97.5 |

### Example 4

### Catalyst B

ZrO₂ extrudates (XZ 16509 from Norton, Process Prod. Corp., Akron (Ohio), USA, 82 m²/g, pore volume 0.28 cm³/g) were crushed to 0.1-1 mm spall. 200 g of the support material were treated with 8 g of Re₂O₇ in 360 ml of water at 100° C. for 2 h and dried at a pressure of 50 mbar. The material was then heated to 300° C. under 50 l (STP)/h of N₂ for 60 min. From an internal temperature of 280° C., 10 l (STP)/h of H₂ were additionally metered in. After 90 min, the H₂ stream was increased to 25 l (STP)/h. The temperature was maintained for a further 2 h, and the precatalyst was afterwards cooled to room temperature in this reducing atmosphere. After purging with 100 l (STP)/h of nitrogen for 1 h, the material was finally passivated with a mixture of 5 l (STP)/h of oxygen in 100 l (STP)/h of N₂ for 2 h. 36.9 g of a platinum nitrate solution (16.25% by weight of Pt) were diluted to 320 ml with water and the precatalyst was saturated with this solution. At 100° C. and a pressure of 50 mbar, the solvent was removed and the finished catalyst reduced. The catalyst contained 2.0% by weight of Re and 2.3% by weight of Pt.

### Examples 5 and 6

25 ml of catalyst B were installed into a continuous tubular reactor. The feed consisted of a maleic acid solution (10% by weight of maleic acid) and was metered in at 25 g/h. Hydrogenation was effected at 160° C. and 80 bar. The liquid effluent was analyzed by means of gas chromatography. The values in table 2 are each quoted as yields in % per mole of maleic acid.

We claim:

1. A catalyst comprising from 0.1 to 20% by weight of rhenium and from 0.05 to 10% by weight of platinum, based on the total mass of the catalyst, on a support, obtainable by a process in which:
a) the optionally pretreated support is treated with a solution of a rhenium compound,
b) dried and heat-treated in a reductive atmosphere at from 80 to 600° C.; and
c) impregnated with a solution of a platinum compound and dried again.

2. A catalyst according to claim 1, wherein the support is selected from the group consisting of: a metal oxide; optionally pretreated activated carbon or a graphitic carbon support; a nitride; a silicide; a carbide; and a boride.

3. A catalyst according to claim 2, wherein the support is selected from the group consisting of: titanium dioxide; zirconium dioxide; hafnium dioxide; and optionally pretreated activated carbon or a graphitic carbon support.

4. A catalyst according to claim 1, wherein the reductive atmosphere comprises at least a portion of at least one gaseous material selected from the group consisting of gaseous ammonia, hydrazine, C₂- to C₆-olefin, carbon monoxide, and hydrogen.

5. A catalyst according to claim 1, wherein, after step b), the catalyst blank obtained is passivated with an oxygenous gas.

6. A catalyst according to claim 1, which is activated by using a reducing gas atmosphere or a liquid reducing agent.

7. A process for preparing alcohols by catalytically hydrogenating carbonyl compounds to alcohols, which comprises using a catalyst according to claim 1.

8. The process according to claim 7, wherein the carbonyl compound is at least one compound selected from the group consisting of aldehydes, carboxylic acids or esters, anhydrides, and lactones.

9. The process according to claim 8, wherein the carbonyl compound is selected from the group consisting of maleic acid, glutaric acid, adipic acid, fumaric acid, succinic acid or esters or anhydrides thereof, and gamma-butyrolactone; and is hydrogenated to 1,4-butanediol.

10. The process according to claim 9, wherein the carbonyl compound is selected from the group consisting of adipic acid, 6-hydroxycaproic acid or esters thereof, caprolactone; and is hydrogenated to 1,6-hexanediol.

11. The process according to claim 7, wherein the hydrogenation is carried out in the liquid phase over solid catalysts at a pressure in the range from 20 to 230 bar and a temperature in the range from 80 to 210° C.

12. The process according to claim 7, wherein the hydrogenation reactor charged with the catalyst is started up under hydrogenation conditions using water or a dilute aqueous solution of the carbonyl compound.

13. The catalyst according to claim 2, wherein the reductive atmosphere comprises at least a portion of at least one gaseous material selected from the group consisting of gaseous amonia, hydrazine, C₂- to C₆-olefin, carbon monoxide, and hydrogen.

14. The catalyst according to claim 3, wherein the reductive atmosphere comprises at least a portion of at least one gaseous material selected from the group consisting of gaseous amonia, hydrazine, C₂- to C₆-olefin, carbon monoxide, and hydrogen.

15. The catalyst according to claim 2, wherein, after step b), the catalyst blank obtained is passivated with an oxygenous gas.

16. The catalyst according to claim 3, wherein, after step b), the catalyst blank obtained is passivated with an oxygenous gas.

17. The catalyst according to claim 4, wherein, after step b), the catalyst blank obtained is passivated with an oxygenous gas.

18. The catalyst according to claim 2, which is activated by using a reducing gas atmosphere or a liquid reducing agent.

19. The catalyst according to claim 3, which is activated by using a reducing gas atmosphere or a liquid reducing agent.

20. The catalyst according to claim 4, which is activated by using a reducing gas atmosphere or a liquid reducing agent.
